(19) United States
(12) Patent Application Publication
(10) Pub. No.: US 2026/0216392 A1
(43) Pub. Date: Jul. 30, 2026

(54) STERILANT STORAGE DEVICE AND STERILIZATION DEVICE COMPRISING SAME

(71) Applicant: PLASMAPP CO., LTD., Daejeon (KR)

(72) Inventor: You Bong LIM, Daejeon (KR)

(73) Assignee: PLASMAPP CO., LTD., Daejeon (KR)

(21) Appl. No.: 19/148,900

(22) PCT Filed: May 14, 2024
(86) PCT No.: PCT/KR2024/006564

§ 371 Date: Jul. 17, 2025

(30) Foreign Application Priority Data
May 25, 2023 (KR) 10-2023-0067941

(51) Int. Cl. A61L 2/26; A61L 2/20

(52) U.S. Cl. CPC A61L 2/26; A61L 2/20; A61L 2202/122

(57) ABSTRACT

The present invention provides a sterilant storage device and a sterilization apparatus including the same, and the sterilant storage device includes a first container storing a sterilant therein, wherein at least a portion of the first container includes a gas-permeable material, a second container enclosing the first container and having a buffer space therein excluding an area where the first container is positioned, an encapsulation member coupled to the second container and enabling to be penetrated by a sterilant extractor that extracts the sterilant, and at least an air filter coupled to the second container and allowing gas to enter and exit the second container.

### TECHNICAL FIELD

The present invention relates to a sterilant storage device and a sterilization apparatus including the same.

### BACKGROUND ART

Most sterilizers used in the medical industry utilize a sterilant aqueous solution of high concentration, and in the sterilization process, the sterilant is vaporized by using heat and the sterilization cycle is performed on an object to be treated by the vaporized sterilant.

Sterilization performance is influenced by various factors, and in particular, the amount and concentration of the sterilant introduced during the sterilization cycle are factors that have a great influence. In order to ensure the reliability of the sterility assurance level (SAL) in the sterilization cycle of a sterilizer, both the amount and the concentration of the sterilant used in the sterilization cycle should be consistently maintained all the time.

However, hydrogen peroxide, which is commonly used as a sterilant, is thermodynamically unstable at room temperature and atmospheric pressure, and thus naturally decomposes over time, to generate gas during the decomposition.

That is, there are concurrent issues in terms of the management of the amount and concentration of the sterilant due to its chemical decomposition and the management of gas generated by the decomposition of the sterilant.

### DISCLOSURE OF INVENTION

### Technical Problem

An object of the present invention is to provide a sterilant storage device configured with a dual structure for regulating internal pressure and including an air filter and a sterilization apparatus including the same.

### Solution to Problem

According to an aspect of the present invention for achieving the above object, there is provided a sterilant storage device including a first container storing a sterilant therein, wherein at least a portion of the first container includes a gas-permeable material, a second container enclosing the first container and having a buffer space therein excluding an area where the first container is positioned, an encapsulation member coupled to the second container and configured to be penetrated by a sterilant extractor that extracts the sterilant. and at least an air filter coupled to the second container and allowing gas to enter and exit the second container.

### Advantageous Effects of Invention

The sterilant storage device according to the present invention is configured with a dual-container structure having a buffer space, thereby stably storing the sterilant. In addition, one or more air filters are provided in the sterilant storage device, which allows appropriate control of the natural decomposition rate of the sterilant and effective discharge of the gas that is generated by the natural decomposition. Through those configurations above, it is possible to prevent the container damage or the sterilant leakage due to the internal pressure increase in the container.

The sterilant storage device according to the present invention is configured with the dual container structure and an asymmetric structure, thereby securing a space through which the gas generated by the natural decomposition flows out to the outside of the container even when the container is tilted or turned over during the storage and transportation of the sterilant, and stably storing the sterilant.

The sterilant storage device according to the present invention may prevent the sterilant from leaking out to the outside of the sterilant storage device in the process of extracting the sterilant. Through those configurations above, it is possible to accurately control the amount of the sterilant that is extracted.

### BRIEF DESCRIPTION OF DRAWINGS

FIG. 1 is a conceptual view illustrating a sterilization apparatus according to an embodiment of the present invention.

FIG. 2 is a perspective view illustrating a sterilant storage device in FIG. 1.

FIG. 3 is a cross-sectional view of the sterilant storage device in FIG. 1.

FIG. 4 is a view illustrating a combination of some components of the sterilant storage device in FIG. 1.

FIG. 5 is an exploded view of the configuration in FIG. 4.

FIG. 6 is a cross-sectional view of the sterilant storage device according to an embodiment of the present invention, showing the sterilant storage device in a tilted state.

FIG. 7 is a cross-sectional view of the sterilant storage device according to another embodiment of the present invention, showing the sterilant storage device in an overturned state.

### BEST MODE FOR CARRYING OUT THE INVENTION

According to an aspect of the present invention, there is provided a sterilant storage device including a first container storing a sterilant therein, wherein at least a portion of the first container includes a gas-permeable material, a second container enclosing the first container and having a buffer space therein excluding an area where the first container is positioned, an encapsulation member coupled to the second container and configured to be penetrated by a sterilant extractor that extracts the sterilant, and at least an air filter coupled to the second container and allowing gas to enter and exit the second container.

In some embodiments, the sterilant storage device may further include a sealing member positioned between the air filter and the second container.

In some embodiments, the sealing member may be formed integrally with the encapsulation member.

In some embodiments, the encapsulation member may include an elastic material, thereby restoring a path penetrated by the sterilant extractor.

In some embodiments, the second container may be configured to control an internal pressure thereof by the air filter, and the gas generated by natural decomposition of the sterilant may enter the buffer space from the first container.

In some embodiments, the first container may include a storage tank storing the sterilant therein and an injection tube connected to the storage tank to allow the sterilant to be injected therethrough into the storage tank.

In some embodiments, the injection tube may protrude outward from the storage tank and be positioned in the buffer space of the second container.

In some embodiments, the injection tube includes a flexible material.

In some embodiments, the storage tank may be formed in a shape that an area of an inner bottom surface is inclined toward a center.

In some embodiments, the first container is positioned offset to one side of the second container therein.

In some embodiments, the second container may include a first body accommodating the first container and a second body coupled to the first body to form an internal space of the second container.

In some embodiments, the encapsulation member and the air filter may be positioned in the second body, and the second container may further include a cover covering the encapsulation member and the air filter arranged in the second body.

According to another aspect of the present invention, there is provided a sterilization apparatus including a sterilant storage device for storing a sterilant, a sterilant extractor extracting the sterilant from the sterilant storage device, and a vaporizer vaporizing the extracted sterilant and supplying the same to an area where an object to be treated is positioned, wherein the sterilant storage device may include a first container storing a sterilant therein, wherein at least a portion of the first container includes a gas-permeable material, a second container enclosing the first container and having a buffer space therein excluding an area where the first container is positioned, an encapsulation member coupled to the second container and configured to be penetrated by a sterilant extractor and at least an air filter coupled to the second container and allowing gas to enter and exit the second container.

In some embodiments, the second container may be configured to control an internal pressure thereof by the air filter, and the gas generated by natural decomposition of the sterilant may enter the buffer space from the first container.

### MODE FOR THE INVENTION

Hereinafter, the configuration and operation of the present invention will be described in detail with reference to embodiments of the present invention illustrated in the accompanying drawings below.

The present invention may undergo various modifications and have various embodiments, and specific embodiments are illustrated in the drawings and described in detail in the following detailed description. The effects and features of the present invention and the methods for achieving them will become apparent from the following detailed description of embodiments, in conjunction with the accompanying drawings. However, the present invention is not limited to the embodiments disclosed herein but may be implemented in various forms.

Hereinafter, embodiments of the present invention will be described in detail with reference to the accompanying drawings, and when describing with reference to the drawings, identical or corresponding elements are designated by the same reference numerals, and redundant descriptions thereof will be omitted.

In the following embodiments, the singular expressions include plural expressions unless the context clearly indicates otherwise.

In the following embodiments, terms such as “include” or “have” merely indicate that the features or elements described in the specification are present, and do not preclude the possibility of presence or addition of one or more other features or elements.

In cases where an embodiment may be implemented differently, specific process steps may be performed in an order different from that described. For example, two processes described in succession may, in practice, be carried out simultaneously or in a reverse order to that which is described.

In the drawings, the size of components may be exaggerated or reduced for convenience of explanation. In other words, the sizes and thicknesses of the components shown in the drawings are presented arbitrarily for convenience of explanation, and therefore, the embodiments described hereinafter are not limited those depicted.

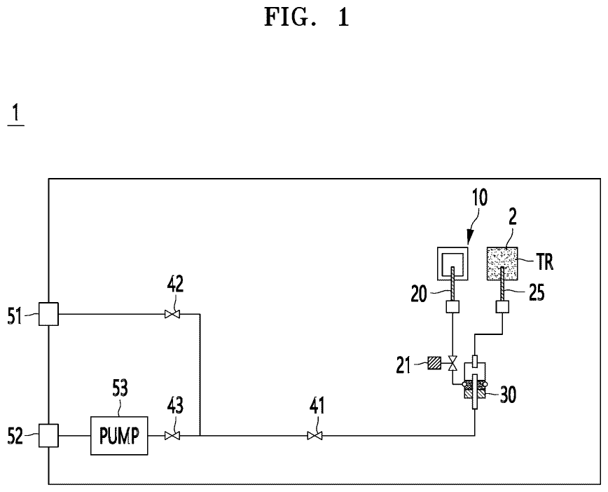

FIG. 1 is a conceptual view illustrating a sterilization apparatus 1 according to an embodiment of the present invention.

Referring to FIG. 1, the sterilization apparatus 1 may include a sterilant storage device 10, a sterilant extractor 20, and a vaporizer 30.

The sterilant storage device 10 may store a sterilant M therein. In this case, the sterilant M may be a liquid-state hydrogen peroxide (H₂O₂), ethylene oxide (C₂H₄O), or Chlorine Dioxide (ClO₂), or the like. The specific configuration of the sterilant storage device 10 will be described below.

The sterilant extractor 20 may penetrate the sterilant storage device 10 and extract the sterilant M stored in the sterilant storage device 10. In addition, the sterilant extractor 20 may deliver the extracted sterilant to the vaporizer 30.

The vaporizer 30 may vaporize the sterilant extracted by the sterilant extractor 20 and deliver the vaporized sterilant to an object to be treated TR contained in the chamber 2.

The sterilization apparatus 1 may further include an extraction valve 21, a sterilant injector 25, a plurality of discharge valves 41, 42, and 43, a plurality of discharge filters 51 and 52 and a pump 53.

The extraction valve 21 may control the flow of the extracted sterilant.

In an embodiment, the extraction valve 21 is positioned between the sterilant extractor 20 and the vaporizer 30, to control the flow of the sterilant extracted by the sterilant extractor 20. The extraction valve 21 may be opened in the extraction process of the sterilant and be closed when the extraction process of sterilant is completed.

The sterilant injector 25 may inject the sterilant vaporized in the vaporizer 30 into the object to be treated TR in the chamber 2. According to an embodiment, the air in the chamber 2 may also be discharged through the sterilant injector 25.

When the sterilization of the object to be treated TR is completed, some of the plurality of discharge valves 41, 42, and 43 may be closed while the remaining discharge valves are opened, and thus the air in the chamber 2 in which the object to be treated is stored may be discharged.

A plurality of filters 51 and 52 may filter the air that is injected into or discharged from the sterilization apparatus 1 through the discharge valve 41, 42, and 43. According to an embodiment, each of the filters 51 and 52 may filter out fine dust, bacteria, and the like.

The pump 53 may apply pressure to discharge the sterilant or air from the chamber 2.

In an embodiment, the pump 53 may discharge air from the chamber 2 for creating a vacuum state inside the chamber 2 before sterilization, or discharge air from the chamber 2 for removing residual sterilant remaining in the chamber 2 after sterilization.

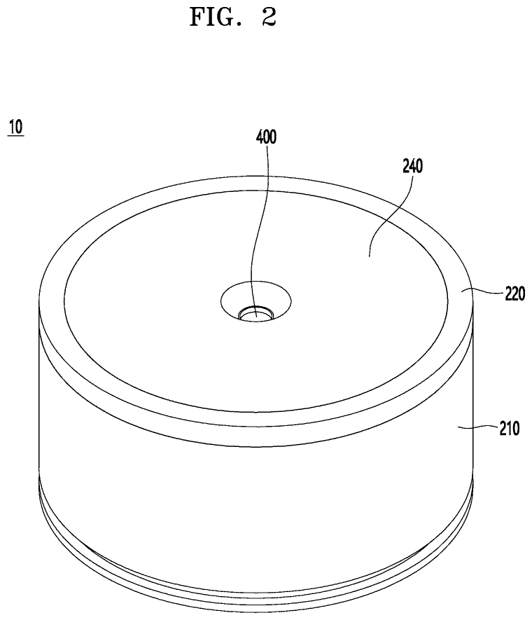

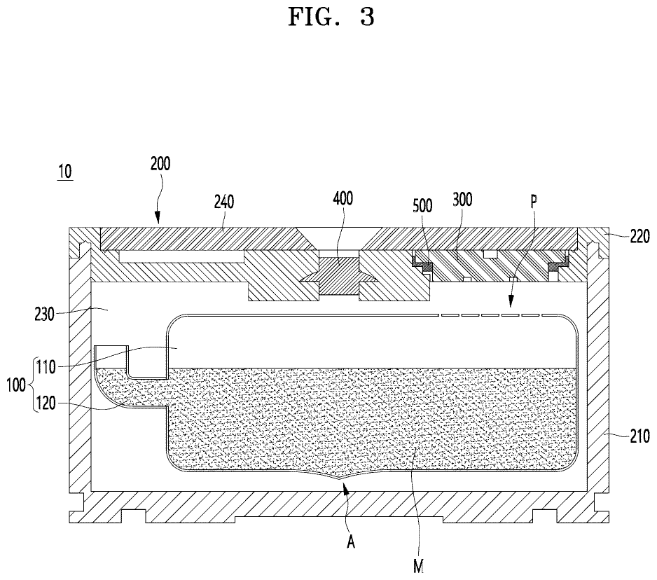

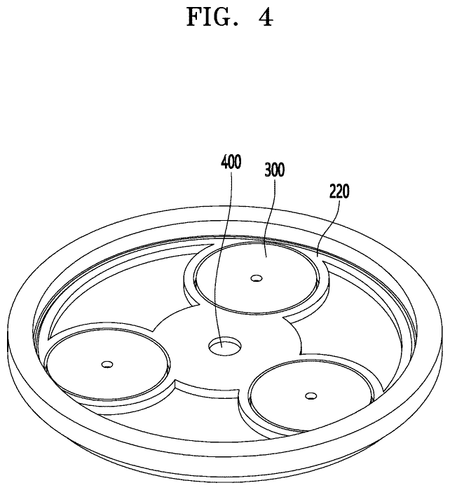

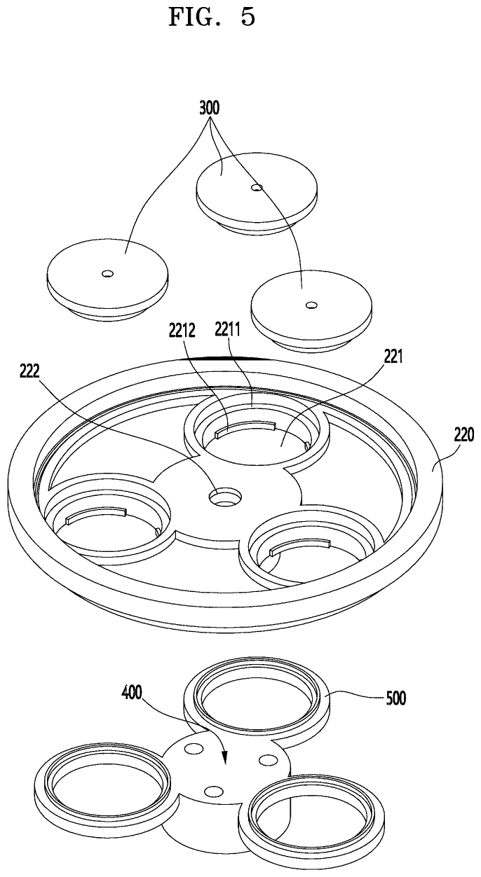

FIG. 2 is a perspective view of the sterilant storage device 10 in FIG. 1 and FIG. 3 is a cross-sectional view of the sterilant storage device 10 in FIG. 1. Furthermore, FIG. 4 is a view illustrating a combination of some components of the sterilant storage device 10 in FIG. 1 and FIG. 5 is an exploded view of the combination in FIG. 4.

Referring to FIGS. 2 to 5, the sterilant storage device 10 according to an embodiment of the present invention may include a first container 100, a second container 200, an encapsulation member 400, and an air filter 300.

The first container 100 may store the sterilant M therein. Particularly, the first container 100 may store the sterilant M of liquid state in a sealed state.

In an embodiment, at least a portion of the first container 100 may include a gas-permeable material. Through at least a portion of the above, the gas generated by the natural decomposition of the sterilant in the first container 100 may flow to the outside of the first container 100.

For example, a portion P of an upper surface of the first container 100 may include the gas-permeable material. However, the position and size of the area formed of the gas-permeable material in the first container 100 are not limited thereto, and at least a portion thereof may be variously configured in such a way that gas in the first container 100 flows to the outside even when the sterilant storage device 10 is tilted or turned over during storage and transportation.

In an embodiment, the gas-permeable material may be polyethylene, polypropylene, or polyolefin. Alternatively, the gas-permeable material may be a combination thereof. However, the gas-permeable material is not limited thereto, and may include various materials through which gas can be physically permeable.

A portion of the rest of the first container 100, excluding the at least portion thereof, may include a non-gas-permeable material.

In an embodiment, the non-gas-permeable material may include a polyamide-based resin such as nylon. Alternatively, the non-gas-permeable material may include a styrene-based resin such as acrylonitrile butadiene styrene (ABS) resin. However, the non-gas-permeable material is not limited thereto and may include various materials through which gas cannot be physically permeable.

In an embodiment, the first container 100 may be positioned offset to one side of the second container 200 therein. Referring to FIG. 3, due to an asymmetrical structure caused by the injection tube 120 that is described later, the first container 100 may be positioned offset to one side of the second container 200 therein.

The first container 100 may include a storage tank 110 and an injection tube 120.

The storage tank 110 may store the sterilant M therein.

In an embodiment, the storage tank 110 may have a cylindrical shape. However, the shape of the storage tank 110 is not limited thereto and may have various shapes capable of storing the sterilant M.

In an embodiment, the storage tank 110 may be formed in such a shape that an area of the inner bottom surface is inclined toward the center. Specifically, an area A of the inner bottom surface of the storage tank 110 may form a well in a concave shape.

In an embodiment, the area A may be positioned in a location corresponding to the encapsulation member 400 that is described later. Through those configurations above, the extraction amount of the sterilant M that is stored in the storage tank 110 may be maximized.

The injection tube 120 may be a path for injecting the sterilant M of liquid state into the storage tank 110. Otherwise, from the storage tank 110, the sterilant M of liquid state may flow into the injection tube 120.

In addition, the injection tube 120 may be a path through which the sterilant of gaseous state that is generated by natural decomposition within the storage tank 110 flows to the outside of the storage tank 110.

The injection tube 120 may be connected to the storage tank 110. For example, as shown in FIG. 3, the injection tube 120 may be connected to a side of the storage tank 110.

In an embodiment, the injection tube 120 may be formed in such a configuration that the injection tube 120 protrudes outward from the storage tank 110 and communicates with the storage tank 110. Specifically, the injection tube 120 may protrude outward from the storage tank 110 and be positioned in the buffer space 230 of the second container 200.

In an embodiment, the injection tube 120 may be made of a flexible material. The shape of the injection tube 120 made of flexible material may be changed. As shown in FIG. 3, the injection tube 120 may be positioned in the second container 200 in such a shape that one end portion is bent with facing upward.

The second container 200 may be formed to enclose the first container 100. The sterilant storage device 10 may be formed with a dual container structure, thereby preventing the leakage of sterilant of liquid or gaseous state.

In an embodiment, as shown in FIG. 2, the second container 200 may have a constant internal space and may be configured to enclose the first container 100 that is positioned within the internal space.

In an embodiment, the second container 200 may be formed correspondently to the shape of the first container 100. For example, the second container 200 may be formed in a cylindrical shape correspondently to the cylindrical first container 100. However, the shape of the second container 200 is not limited thereto and may take various forms capable of enclosing the first container 100.

The second container 200 may have the buffer space 230, excluding the area where the first container 100 is positioned, within the internal space.

The gas generated by the natural decomposition of the sterilant M of liquid state may enter the buffer space 230 from the inside of the first container 100. The internal pressure of the second container 200 may be determined according to the amount of the sterilant of gaseous state that enters the buffer space 230. The control of the internal pressure of the second container 200 will be described in detail below.

The second container 200 may include a first body 210 and a second body 220.

The first body 210 may accommodate the first container 100. Specifically, the first container 100 may be positioned inside the first body 210. In addition, as described above, the first container 100 may be positioned offset to one side of the first body 210 therein.

The first body 210 may have a form in which one side is open. For example, as shown in FIGS. 2 and 3, the first body 210 may have a form in which an upper surface is open. However, the open surface is not limited thereto and may be any other areas where the first container 100 is positioned within the first body 210.

In an embodiment, the first body 210 may have a cylindrical shape in which the upper surface is open, as shown in FIGS. 2 and 3. However, the shape of the first body 210 is not limited thereto and may take various forms capable of accommodating the first container 100 therein.

The second body 220 may be coupled to the first body 210. Specifically, the second body 220 may be coupled to the first body 210 to form the internal space of the second container 200.

For example, as shown in FIGS. 2 and 3, the second body 220 may have a disc shape. In this case, the second body 220 may be coupled to the upper portion of the cylindrical first body 210 having an open upper surface. However, the shape of the second body 220 is not limited thereto and may be determined together with the shape of the first body 210.

Specifically, referring to FIG. 5, the second body 220 may include a first insertion hole 221 and a second insertion hole 222.

The air filter 300, which will be described later, may be inserted and positioned in the first insertion hole 221 of the second body 220.

In an embodiment, a plurality of first insertion holes 221 may be formed in the second body 220, and specifically, the number of first insertion holes 221 corresponding to the number of air filters 300 may be formed. For example, when the sterilant storage device 10 includes three air filters 300 as shown in FIG. 5, the second body 220 may have three first insertion holes 221.

A sealing member 500 may be coupled to and positioned in the first insertion hole 221. By coupling the sealing member 500 to the first insertion hole 221, the gas may be prevented from leaking out to areas other than the air filter 300.

The first insertion hole 221 may be formed into a stepped structure 2211 with different diameters, enabling it to support the air filter 300 inserted into the first insertion hole 221. Specifically, the air filter 300 may be inserted into the first insertion hole 221 from the upper side of the second body 220 and supported by the stepped structure 2211, so that the air filter 300 may be stably placed in the first insertion hole 221.

The second body 220 may have a protrusion 2212 protruding from the inside of the first insertion hole 221.

In an embodiment, the protrusion 22121 may be engaged with the sealing member 500 that is coupled to the first insertion hole 221, thereby supporting the sealing member 500. Specifically, as will be described later, the sealing member 500 may be positioned between the air filter 300 and the first insertion hole 221, and in this case, it may be supported by the protrusion 2212.

In an embodiment, the protrusion 2212 may be formed to protrude from an inner surface of the first insertion hole 221. However, the location of the protrusion 2212 is not limited thereto and may be formed in various positions at which the sealing member 500 is supported.

In an embodiment, a plurality of protrusions 2212 may be arranged in a single first insertion hole 221. For example, as shown in FIG. 5, three protrusions 2212 may be arranged spaced apart from each other in a single first insertion hole 221. By adjusting the number of protrusions 2212, the coupling force and airtightness of the sealing member 500 may be improved.

The second insertion hole 222 may be formed in an area of the second body 220 and may perform the function of guiding the inserted sterilant extractor 20 into the inside of the first container 100. For example, the second insertion hole 222 may be formed in the central area of the second body 220, but is not necessarily limited thereto, and may be formed in any area capable of guiding the sterilant extractor 20 to the first container 100.

The second container 200 may further include a cover 240. The cover 240 may cover the encapsulation member 400 and the air filter 300 that are arranged on the second body 220.

In an embodiment, at least a portion of the cover 240 may include a gas-permeable material. The gas passing through the air filter 300 may be discharged to the outside of the cover 240 through an area that includes the gas-permeable material.

In another embodiment, although not shown in FIG. 2, the cover 240 may include a hole through which the gas enters and exits. However, the configuration of the cover 240 is not limited to this and may be in various forms that allow the gas to enter and exit.

The air filter 300 may provide a path through which the gas enters and exits the second container 200.

The air filter 300 may be coupled to the second container 200. Specifically, the air filter 300 may be coupled to the second body 220 of the second container 200. In this case, through the air filter 300, the gas within the second container 200 may be discharged to the outside of the second container 200.

In an embodiment, a plurality of air filters 300 may be coupled to the second container 200. For example, as shown in FIG. 4, three air filters 300 may be coupled to the second body 220 of the second container 200.

Specifically, the air filter 300 may provide a path through which the gas flows, but it may act as a resistance when the gas flows therethrough, thereby limiting the flow rate of the gas. Therefore, the internal pressure of the second container 200 may be controlled by adjusting the number of air filters 300.

For example, when the internal pressure of the second container 200 is to be relatively increased to relatively slow down the natural decomposition of the sterilant M, only one air filter 300 may be arranged. In addition, when the internal pressure of the second container 200 is to be relatively lowered so as to stably store the sterilant M, two or more air filters 300 may be arranged.

In an embodiment, a plurality of air filters 300 may be arranged spaced apart from each other in the second container 200. For example, as shown in FIG. 4, three air filters 300 may be arranged at regular intervals in the second body 220. However, the arrangement of the plurality of air filters 300 is not limited thereto.

The encapsulation member 400 may be arranged in the second insertion hole 222 of the second container 200 and perform the function of encapsulating the second insertion hole 222. In this case, the encapsulation member 400 may be penetrated by the sterilant extractor 20.

In an embodiment, the encapsulation member 400 may include an elastic material. The encapsulation member 400 formed of the elastic material may restore the path penetrated by the sterilant extractor 20. Through those configurations above, the sterilant storage device 10 may prevent the leakage of the sterilant and accurately control the amount of sterilant stored.

The sterilant storage device 10 may further include the sealing member 500. The sealing member 500 may prevent the gas from leaking out from an area other than the air filter 300 coupled to the second container 200.

A sealing member 500 may be arranged between the air filter 300 and the second container 200. Specifically, the sealing member 500 may be arranged between the air filter 300 and the first insertion hole 221 of the second body 220. The arrangement and coupling method of the sealing member 500 will be described in detail below.

In an embodiment, the sealing member 500 may be formed integrally with the encapsulation member 400. FIG. 5 illustrates an embodiment in which the sealing member 500 is formed integrally with the encapsulation member 400. In this case, the sealing member 500 may include an elastic material such as the encapsulation member 400.

The sterilant storage device 10 may simplify its configuration by integrally forming the sealing member 500 and the encapsulation member 400 and may more effectively prevent the gas or the sterilant from leaking through areas other than the air filter 300.

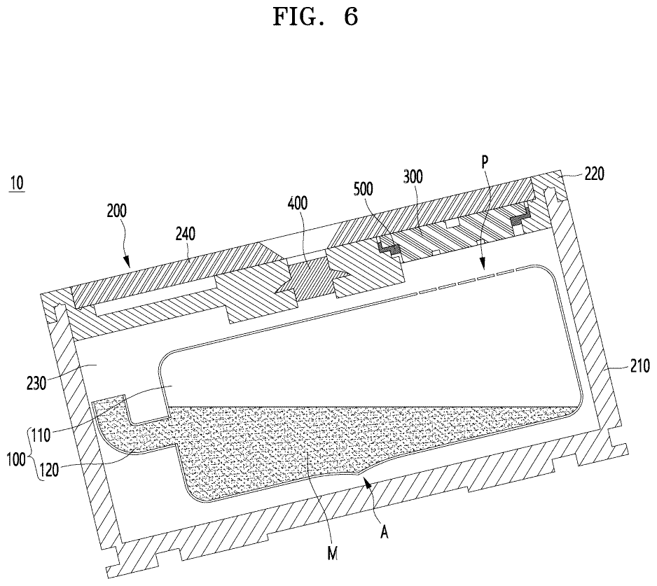

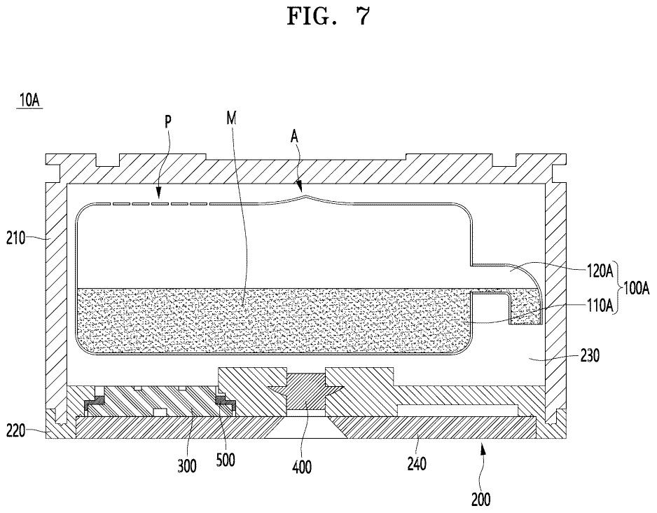

FIG. 6 is a cross-sectional view of the sterilant storage device 10 according to an embodiment of the present invention, showing the sterilant storage device 10 in a tilted state, and FIG. 7 is a cross-sectional view of the sterilant storage device 10A according to another embodiment of the present invention, showing the sterilant storage device 10A in an overturned state.

Referring to FIG. 3 together, the sterilant stored in the first container 100 may naturally decompose and exist in gaseous state within the sterilant storage device 10.

In an embodiment, since at least a portion of the first container 100 includes the gas-permeable material, the sterilant, which is generated by the natural decomposition and in gaseous state in the first container 100, may flow out to the outside of the first container 100.

When the injection tube 120 includes the gas-permeable material, the gas, which is generated by the natural decomposition of the sterilant M in the first container 100, may flow to the outside of the first container 100 through the injection tube 120.

The gas flowing to the outside of the first container 100 may be positioned at first in the buffer space 230 within the second container 200. The internal pressure of the second container 200 may be determined according to the amount of the gas within the buffer space 230. As the pressure inside the second container 200 increases, it becomes relatively more difficult for the gas within the first container 100 to flow outward, so the natural decomposition rate of the sterilant M may become relatively slower.

The gas within the buffer space 230 may be discharged to the outside of the sterilant storage device 10 through the air filter 300 coupled to the second container 200. That is, the internal pressure the sterilant storage device 10 may be controlled as the gas is discharged through the air filter 300.

Referring to FIGS. 6 and 7, even when the sterilant storage device 10 is tilted or turned over, the gas within the sterilant storage device 10 may be discharged to the outside through at least one air filter 300 via the buffer space 230 in the second container 200. Through those configurations above, the internal pressure of the sterilant storage device 10 may be controlled to prevent damage to the container and stably store the sterilant M.

As described above, the sterilant storage device 10 may include the first container 100 and the second container 200 enclosing the first container 100. Even if the sterilant storage device 10 is tilted as shown in FIG. 6 or the sterilant storage device 10A is turned over as shown in FIG. 7 in the storage or the transportation process, the sterilant M of liquid state remains in the first container 100 due to the dual container structure, and thus the air filter 300 is not clogged.

Accordingly, even if the sterilant storage device 10 is tilted or turned over, the gas generated by the natural decomposition of the sterilant M is discharged to the outside of the sterilant storage device 10 through the buffer space 230 and the air filter 300, thereby controlling the internal pressure thereof.

As described above, the first container 100 may be positioned offset to one side of the second container 200 therein. Through those configurations above, even when the sterilant storage device 10 is tilted or turned over, the gas within the first container 100 may flow into the buffer space 230, and then be discharged to the outside of the sterilant storage device 10 through at least one of the plurality of air filters 300.

As described above, the first container 100 may have an asymmetrical structure including the deformable injection tube 120 on one side. When the sterilant storage device 10 is tilted in the direction where the injection tube 120 is positioned, some of the sterilant M of liquid state may flow into the injection tube 120.

Through this asymmetric structure, even when the sterilant storage device 10 is tilted or turned over, the gas within the first container 100 may flow into the buffer space 230, and then the gas may be discharged to the outside of the sterilant storage device 10 through at least one of the plurality of air filters 300.

As described above, the area P that includes the gas-permeable material in the first container 100 may be configured in various ways. FIG. 7 shows the sterilant storage device 10A in which a portion of the lower surface of the first container 100A includes the gas-permeable material. Even if the sterilant storage device 10A is turned over, the gas within the first container 100A may flow into the buffer space 230 due to the structure described above.

Therefore, by forming a plurality of areas of the first container 100 with the gas-permeable material, the gas may always flow to the outside of the first container 100, even when the sterilant storage device 10 is tilted or turned over in any direction. In this case, the gas generated within the first container 100 may be discharged to the outside of the sterilant storage device 10 through the buffer space 230 and the air filter 300.

As described above, the present invention has been described with reference to the embodiments shown in the drawings, but these are merely illustrative, and it will be understood that by those skilled in the art that various modifications and equivalent alternative embodiments may be derived from this disclosure. Therefore, the true technical protection scope of the present invention should be defined by the spirit of the invention as set forth in the appended claims.

The specific implementations described in the embodiments are merely exemplary and should not be construed as limiting the scope of the embodiments in any way. In addition, unless explicitly stated as “essential” or “importantly”, or similar terms, the component or element may not be necessarily required for the implementation of the present invention.

In the specification of the embodiment (particularly in the claims), the use of terms such as “above” and similar referential expressions may refer to both singular and plural forms. In addition, when a range is described in the embodiment, it is to be understood that the invention includes implementation using individual values within the stated range (unless otherwise specified), and such disclosure is equivalent to individually describing each value within that range in the detailed description. Finally, unless the steps constituting the method according to the embodiment are explicitly described in a specific order or otherwise stated to the contrary, the steps may be performed in any suitable order. The embodiments are not necessarily limited to the order in which the steps are described. The use of all examples or exemplary terms (such as “for example,” “etc.”) in the embodiments is merely for the purpose of describing the embodiments in detail, and unless otherwise limited by the claims, such examples or exemplary terms should not be construed as limiting the scope of the embodiments. Furthermore, those skilled in the art will appreciate that various modifications, combinations and variations may be made according to design conditions and factors within the scope of the appended claims or their equivalents.

### INDUSTRIAL APPLICABILITY

According to an embodiment of the present invention, there are provided a sterilant storage device and a sterilization apparatus including the same. In addition, the embodiments of the present invention may be applied to technologies such as those for storing and using a sterilant.

1-14. (canceled)

15: A sterilant storage device comprising:
a first container storing a sterilant therein, wherein at least a portion of the first container includes a gas-permeable material;
a second container formed to enclose the first container; and
at least an air filter coupled to the second container and allowing only gas to enter and exit the second container.

16: The sterilant storage device of claim 15, wherein
the second container has a buffer space therein excluding an area where the first container is positioned.

17: The sterilant storage device of claim 16, wherein
the gas contained in the buffer space of the second container is discharged to an outside of the second container through the air filter.

18: The sterilant storage device of claim 17, wherein
the first container is positioned offset to one side of the second container therein, to allow the gas contained in the buffer space to enter and exit through the air filter.

19: The sterilant storage device of claim 15, wherein
the second container includes:
a first body accommodating the first container; and
a second body coupled to the first body to form an internal space of the second container,
wherein an area where the first body and the second body are coupled to each other is sealed.

20: The sterilant storage device of claim 19, further comprising:
a sealing member positioned between the air filter and the second container and sealing an area where the air filter and the second container are coupled to each other.

21: The sterilant storage device of claim 15, further comprising:
an encapsulation member coupled to the second container and configured to be penetrated by a sterilant extractor that extracts the sterilant.

22: The sterilant storage device of claim 21, wherein
the encapsulation member includes an elastic material.

23: The sterilant storage device of claim 22, wherein
the encapsulation member is elastically deformed in such a way that the second container is encapsulated while the sterilant extractor is penetrated through the encapsulation member.

24: The sterilant storage device of claim 22, wherein
the encapsulation member is elastically deformed in such a way that the second container is encapsulated when the sterilant extractor penetrating the encapsulation member is separated from the encapsulation member.

25: The sterilant storage device of claim 21, wherein
the encapsulation member extends in a height direction of the first container and is positioned adjacent to a virtual axis passing through a center thereof.

26: A sterilization apparatus comprising:
a sterilant storage device for storing a sterilant;
a sterilant extractor extracting the sterilant from the sterilant storage device; and
a vaporizer vaporizing the extracted sterilant and supplying the same to an area where an object to be treated is positioned,
wherein the sterilant storage device includes:
a first container storing a sterilant therein, wherein at least a portion of the first container includes a gas-permeable material;
a second container formed to enclose the first container; and
at least an air filter coupled to the second container and allowing only gas to enter and exit the second container.

27: The sterilization apparatus of claim 26, wherein
the second container has a buffer space therein excluding an area where the first container is positioned.

28: The sterilization apparatus of claim 27, wherein
the sterilant storage device further includes:
an encapsulation member coupled to the second container and configured to be penetrated by the sterilant extractor.

29: The sterilization apparatus of claim 28, wherein
the sterilant extractor extends in a height direction of the sterilant storage device and penetrates through the encapsulation member, and is inserted into an inside of the first container to extract the sterilant.

30: The sterilization apparatus of claim 26, wherein
the first container is formed in such a shape that an area of an inner bottom surface thereof is inclined toward a center corresponding to a shape of the sterilant extractor.
